(19) United States
(12) Patent Application Publication
(10) Pub. No.: US 2011/0071227 A1
(43) Pub. Date: Mar. 24, 2011

(54) RAPIDLY WETTING MATERIAL CONTAINING HYDROCOLLOID, METHOD FOR THE MANUFACTURE THEREOF AND USE THEREOF

(75) Inventors: Michael Ahlers, Eberbach (DE); Steffen Oesser, Glucksburg (DE); Michael Grzinia, Schwaikheim (DE); Klaus Flechsenhar, Eberbach (DE)

(73) Assignee: GELITA AG, Eberbach (DE)

(21) Appl. No.: 12/897,255

(22) Filed: Oct. 4, 2010

Related U.S. Application Data
(63) Continuation of application No. PCT/EP2009/054452, filed on Apr. 15, 2009.

(30) Foreign Application Priority Data
Apr. 15, 2008 (DE) 10 2008 020 197.9

(51) Int. Cl. A61K 47/42; B01J 20/00; B01J 20/24; B01J 20/22

(52) U.S. Cl. 514/774; 502/400; 502/404; 502/403

(57) ABSTRACT

The present invention relates to a method for the production of a quickly wettable material containing natural hydrocolloid in the form of a shaped body, in which a material containing natural hydrocolloid in the form of a shaped body is exposed to a plasma.

The present invention relates to a quickly wettable material containing natural hydrocolloid in the form of a shaped body. The invention additionally relates to a method for the production of this material as well as the use of the material in different fields of application.

A series of different polymer substances, which are generally of natural origin (in particular polysaccharides and proteins), but are also synthetically produced in part, are referred to as hydrocolloids. They are distinguished by the property of forming gels or viscous solutions in aqueous systems, wherein some hydrocolloids are water-soluble (as a function of the temperature), while others are present in the form of colloidal solutions, dispersions or emulsions. These and other properties of the hydrocolloids can be influenced in some cases by means of the degree of hydrolysis, e.g. in the production of soluble gelatin from insoluble collagen.

Because of these properties as well as their physiological safety, natural hydrocolloids are widely used in the production of foodstuffs and cosmetics as thickening agents or stabilisers, i.e. to adjust a specific consistency or texture of the products. Moreover, hydrocolloids or materials containing hydrocolloid are also used in medicine, e.g. in wound care or as substrate for living cells, wherein the good physiological compatibility, biodegradability and gel-forming properties of many hydrocolloids can also be advantageously used here.

A problem that occurs in the use of hydrocolloids in all the above-mentioned fields is their poor or low wettability. The cause of this phenomenon is presumably that drying must generally be conducted during the course of the production or extraction of hydrocolloids, wherein hydrophobic areas of the actually hydrophilic hydrocolloid molecules in contact with the air are oriented towards the air, so that the particles, agglomerates or shaped bodies formed have a hydrophobic surface.

The consequence of this poor wettability is that in the field of foodstuffs, for example, a longer time, intense agitation or a higher temperature is necessary for dissolving or dispersing a hydrocolloid powder than would be the case with a quickly wettable product. In the industrial field this leads to higher costs, whereas the ease of handling the product (e.g. gelatin powder) is reduced at the end user.

For different medical applications in particular hydrocolloids are used in the form of shaped bodies. Materials containing hydrocolloid that are quickly wettable on contact with aqueous media, in particular body fluids, are also fundamentally desirable here.

To solve this problem, a method of the aforementioned type is proposed according to the invention, in which a material containing hydrocolloid in the form of a shaped body is exposed to a plasma.

It has been surprisingly found that by treating a material containing hydrocolloid with a plasma, i.e. an at least partially ionised gas, its wettability can be improved quite considerably, so that the resulting material is often completely wettable in a very short time. However, at the same time the advantageous properties of the hydrocolloids remain substantially uninfluenced, in particular even the pH value on the surface of the material containing hydrocolloid remains substantially unchanged. This indicates that in spite of the extreme conditions that a plasma represents, chemical modification of the hydrocolloid evidently only occurs to a small extent, but this is nevertheless sufficient to significantly improve the wettability.

### Materials Containing Hydrocolloid

In principle, any material containing natural hydrocolloid, in which an improvement of the wettability is necessary, can be used for the method according to the invention. In the sense of the present invention, natural hydrocolloids are understood to be those hydrocolloids that are substantially based on polymers of natural origin, wherein this does not exclude an additional modification of natural polymers in order to provide these with hydrocolloidal properties (such as e.g. in the case of cellulose derivatives). The hydrocolloid can be selected in particular from the group comprising collagen, gelatin, casein, whey proteins, hyaluronic acid, starch, cellulose, pectins, carrageens, chitosan, agar, alginates, hydrolysates thereof, derivatives thereof as well as mixtures with one another.

The material containing hydrocolloid is preferably composed of hydrocolloid in predominant proportions, i.e. to more than 50% by weight. Even if further, possibly water-soluble components are contained in the material, a deficient wettability can result because of the proportion of hydrocolloid. Further components that can preferably be contained in the material containing hydrocolloid are specified in detail below in association with particular embodiments of the invention.

In preferred embodiments of the invention, the material comprises more than approximately 80% by weight, preferably more than approximately 90% by weight of hydrocolloid. In particular, the material can also be composed substantially completely of hydrocolloid.

It should be noted regarding the composition of the material that the hydrocolloid can contain a specific proportion of water, which is bound by the hydrocolloid in equilibrium with the ambient air. The material containing hydrocolloid can be used in this form for the method according to the invention. Alternatively, the water content of the hydrocolloid can be reduced by drying before the plasma treatment, as a result of which an increased liquid absorption capacity of the produced material can be achieved in some cases.

To conduct the method according to the invention, the material containing hydrocolloid can be present in the form of a powder, granular material or agglomerate. Hydrocolloids in such a form are used as raw materials in particular in the fields of foodstuffs and cosmetics. As a result of the method according to the invention, corresponding powders, granular materials or agglomerates can be produced that are quickly wettable and can therefore be processed more quickly and more inexpensively.

The material containing hydrocolloid is provided as a shaped body according to the invention. In the sense of the present invention a shaped body has a predetermined spatial structure, in contrast to a powder for instance. The shaped bodies can be solid or hollow or have a cellular structure. Some examples of shaped bodies comprise films, sponges, woven fabrics, nonwovens, tubes, particles or spheres. Preferred embodiments of such shaped bodies and their use are described in detail further below.

### Plasma Treatment

In the method according to the invention the plasma is preferably a low-pressure plasma, i.e. a plasma having a pressure that lies significantly below the normal pressure of 1013.25 mbar. In particular, the method can be conducted with a low-pressure plasma of approximately 1 mbar or less, preferably approximately 0.5 mbar or less, further preferred approximately 0.2 mbar or less.

Advantageously, the plasma is an O₂ plasma and/or H₂O plasma. The generation of corresponding plasmas is known from the state of the art.

The method is preferably undertaken such that the material containing hydrocolloid is exposed to the plasma in a closed space, in which the plasma is generated. The method according to the invention can be conducted in particular in a commercially available plasma plant.

The time duration, for which the material containing hydrocolloid is exposed to the plasma, can be varied depending on the type of hydrocolloid as well as the structure and size of the material to obtain the desired effect in terms of wettability. In most cases, the material should be exposed to the plasma for longer than approximately 1 minute in order to obtain a noticeable effect. The time duration preferably amounts to approximately 5 to approximately 180 minutes, further preferred approximately 20 to approximately 60 minutes.

The method according to the invention is conducted so that at least a part of the surface of the material containing hydrocolloid is exposed to the plasma. However, the entire surface is preferably exposed to the plasma.

### Materials Containing Gelatin

In a preferred embodiment of the invention, the hydrocolloid comprises gelatin. Gelatin not only plays a dominant role among hydrocolloids in the production of foodstuffs, but is also suitable in a special way as a starting product for different materials for application in medicine.

Especially with respect to the medical application, gelatine with an appropriate purity is distinguished by a very good tissue and cell compatibility and a substantially complete biodegradability. These properties remain substantially uninfluenced by the plasma treatment according to the invention. In particular, the plasma-treated material has very good cell compatibility and no measurable change in pH value occurs on the surface, which is important in the use of materials containing gelatin as substrate for living cells, for example.

The gelatin used for the method according to the invention can be chemically modified. In particular, the modification can consist of providing individual amino acid side chains with additional functional groups in order to increase or reduce the affinity of the material containing gelatin in relation to specific tissue or cell types.

According to a further embodiment of the invention, the gelatin is at least partially cross-linked. By cross-linkage of the gelatin, which can be achieved chemically and/or enzymatically, this is changed into an insoluble form, so that the corresponding materials containing cross-linked gelatin do not immediately dissolve upon contact with an aqueous medium at elevated temperature (e.g. 37° C.), but are only hydrolytically decomposed over a certain period of time. The length of this decomposition time can be varied by means of the degree of cross-linking.

Such a cross-linkage for reducing the solubility of the material can also be undertaken analogously in other water-soluble hydrocolloids.

Interestingly, it has been found that while the wettability of the hydrocolloid is improved by the plasma treatment according to the invention, the solubility of the material and the decomposition behaviour of the shaped body formed therefrom is not or not significantly influenced.

### Gelatin Sponges

A particular aspect of the present invention relates to the application of the method in a material containing gelatin, which has a cellular structure. Such materials with a cellular structure are referred to below as sponges.

Gelatin sponges are used in medicine in particular for the promotion of haemostasis, i.e. for stopping haemorrhages (e.g. as wound dressing or in operations) or as a substrate for living cells (e.g. in the production of tissue implants). In all these cases, a complete wetting and hydration of the sponge is desirable before or on application, and therefore quickly wettable gelatin sponges that are produced by means of the method according to the invention can be used considerably more effectively.

Because of their poor wettability, the gelatin sponges known from the state of the art must be moistened by immersing in water before they can be used as wound dressing, for example, since they have a noticeable absorbency only in moist state. In comparison, gelatin sponges that have been produced using the method according to the invention in dry state already have an astonishingly high absorbency with respect to blood. The absorption capacity compared to non-plasma-treated gelatin sponges is also significantly higher, i.e. a larger quantity of blood is absorbed overall over the course of time.

Gelatin sponges as such can be produced by the method described in patent document DE 10 2004 024 635 A1, for example, in which an aqueous gelatin solution is foamed and then dried. A cross-linkage of the gelatin can occur in this case by adding a cross-linking agent to the gelatin solution and/or by the action of a cross-linking agent on the produced sponge. The gelatin sponge can contain further components besides this such as e.g. softeners.

The gelatin sponges can be provided in different shapes depending on the desired site of application. Preferred shapes are e.g. cuboids with different dimensions as wound dressing or for operations, cylinders for using as tampons, sponge films for dressing large-surface wounds or sponges in the form of spheres.

The cellular structure of the gelatin sponges preferably has an average pore diameter of less than approximately 300 μm. Pore diameters in this range are well suited for the aforementioned applications, in particular also for colonising the sponge with living cells.

The method according to the invention preferably comprises a mechanical compression of the material containing gelatin. As a result of this compression of a gelatin sponge that is brittle in dry state, a partial breaking open of the cellular structure is effected and an open-pored material is obtained. This breaking open of the cellular structure occurs without the structural cohesion of the sponge being affected and it losing its integrity. In this way, in conjunction with the plasma treatment, by means of which not only the wettability of the cellular structure is improved, but the entire inside surface of the cellular structure is preferably also increased, a gelatin sponge can be produced that quickly absorbs an aqueous medium on contact therewith and is completely wetted and hydrated.

The mechanical compression of the material can be conducted both before and after the plasma treatment. However, it is preferred if the material is compressed before it is exposed to the plasma, since in this case the plasma treatment is more effective with respect to the inside surface of the cellular structure.

To achieve a noticeable effect as a result of the mechanical compression, the gelatin sponge is preferably compressed in at least one direction by approximately 40% or more, further preferred by approximately 50% or more. Compression in only one direction, e.g. by rolling the material, is frequently sufficient, but a compression of the material in multiple directions is also possible.

During wetting and hydration of a compressed, plasma-treated gelatin sponge the compression is partly reversed, i.e. the sponge expands when absorbing the aqueous medium. This effect can be used to produce gelatin sponges, which are not only quickly wetted on contact with liquids, but also increase their volume to a considerable extent, in some instances to their original size before compression. In this case, it is preferred if the material is compressed in at least one direction by approximately 65% or more, further preferred by approximately 75% or more, during the course of the method according to the invention.

Such highly compressed, quickly wettable gelatin sponges are not only able to absorb particularly large quantities of blood or other body fluids, but can also assume a supporting or closing function for the surrounding tissue. Particularly in postoperative wound care, such gelatin sponges provide excellent conditions in postoperative wound care compared to non-resorbable materials, since because of the biodegradability of gelatin no further intervention is necessary to remove the material.

Quickly wettable gelatin sponges with high swelling capacity can also be used particularly advantageously as nasal plugs, i.e. both for emergency care in the case of severe cases of nosebleeds and during operations in the nasal area (in particular in functional endoscopic sinus surgery as well as turbinoplasty). With the known indications relatively large quantities of blood have to be absorbed as quickly as possible.

In order to assure as even a swelling of the gelatin sponge as possible, the compression can occur in multiple directions (e.g. a radial compression can be performed in the case of a cylindrical sponge, in particular a nasal plug).

A further surgical procedure, in which postoperative haemorrhages occasionally occur, is a tonsillectomy (removal of the tonsils). In this case, such haemorrhages can also be dressed by means of quickly wettable gelatin sponges.

Gelatin sponges with a higher or lower hardness in the hydrated state can be desirable, depending on the area of use. The softest possible materials are preferred, for example, with a view to preventing scarring in the case of functional endoscopic sinus surgery, whereas in turbinoplasty gelatin sponges with a slightly greater firmness are advantageous to exert a certain pressure on the nasal artery. The hardness of the gelatin sponges in hydrated state can be influenced in particular by the density of the material as well as the cellular structure.

In a further embodiment of the invention, the material containing gelatin comprises one or more pharmaceutical active substances, which can be selected in accordance with the application case. To further improve the haemorrhage-controlling effect, gelatin sponges can comprise haemostatics in particular.

The gelatin sponges can be additionally coloured to make differentiation between different products easier for the user. This applies equally to other materials containing gelatin, which are described below.

### Gelatin Films

In a further embodiment of the invention, the method is conducted with a material containing gelatin in the form of a film. Such gelatin films are also used in the medical field, e.g. as substrate for living cells or in the production of tissue implants.

Preferred gelatin films contain softeners such as glycerine, for example, to increase the flexibility of the films in dry state. However, the problem of poor wettability also arises when inserting these dry films into aqueous media and this can be overcome by application of the method according to the invention.

The gelatin films preferably have a thickness of approximately 20 to approximately 500 μm, preferably approximately 50 to approximately 100 μm.

A preferred method for the production of gelatin films is described in patent document DE 10 2004 024 635 A1.

### Gelatin Nonwovens

According to a further aspect of the invention, a material containing gelatin in the form of a matted fibre scrim is used to conduct the method.

Matted fibre scrims based on gelatin, which can also be referred to as nonwovens, can be used in part as an alternative to gelatin sponges for the aforementioned purposes. The gelatin nonwovens change into a closed-pored fibrous gel structure in the hydrated state, which also provides good conditions in particular as a substrate for living cells. Moreover, such nonwovens can also be used for cosmetic purposes, in particular as face masks.

The fibres of the matted scrim preferably have an average thickness in the range of approximately 1 to approximately 500 μm, further preferred in the range of approximately 3 to approximately 200 μm, in particular in the range of approximately 5 to approximately 100 μm.

A method for the production of gelatin nonwovens by means of a rotary spinning process is described in German patent application No. 10 2007 011 606.

### FURTHER ASPECTS OF THE INVENTION

The present invention additionally relates to a quickly wettable material containing natural hydrocolloid in the form of a shaped body, which is obtainable using the above-described method. Particular advantages and embodiments of the material according to the invention have already been explained in association with the method according to the invention.

Moreover, the material according to the invention can be defined by means of its initial wettability, which falls at the rate at which a specific quantity of an aqueous medium is absorbed by the material.

According to a preferred embodiment, the invention relates to a quickly wettable material containing hydrocolloid with a cellular structure, wherein the material has such an initial wettability that it absorbs 50 μl of an aqueous medium within 10 seconds at maximum, in particular 5 seconds at maximum. The material containing hydrocolloid is in particular a material containing gelatin in the form of a gelatin sponge.

In addition, this material preferably has such a swelling capacity that when immersed in water it increases its volume by at least 200% within 6 seconds at maximum. Such a material can be obtained in particular by previous compression of the material with the cellular structure.

The measurement of the initial wettability and the increase in volume are described in detail in the following examples.

The invention additionally relates to the use of a material containing hydrocolloid according to the invention in the production and/or processing of foodstuffs, as stated above.

The invention additionally relates to the use of the material according to the invention in human or veterinary medicine, in particular for promoting haemostasis (i.e. stopping haemorrhages), as wound dressing and/or as nasal plug, as well as a substrate for living cells, and/or for the production of tissue implants. Reference is likewise made to the above description of the method according to the invention for details of the respective uses.

These and further advantages of the invention are explained in more detail on the basis of the following examples with reference to the figures.

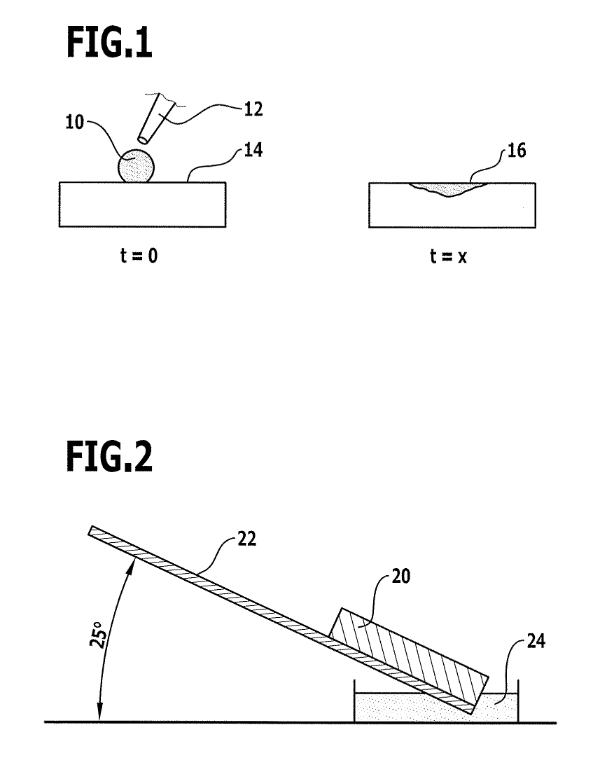

FIG. 1 is a schematic representation of a test for determining the initial wettability;

FIG. 2 is a schematic representation of a test for determining the rate of blood absorption;

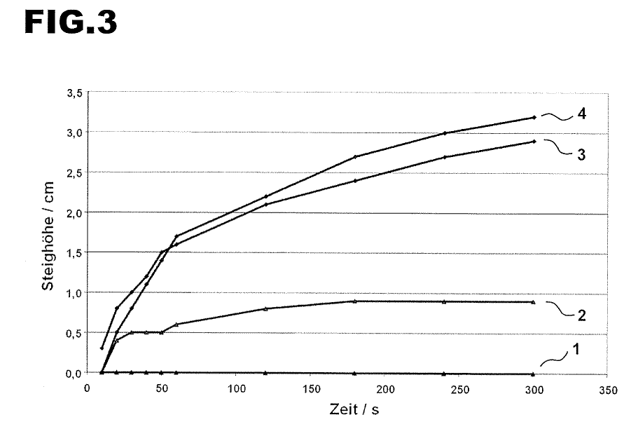

FIG. 3 is a graph relating to the rate of blood absorption by plasma-treated gelatin sponges (according to the invention) and also by non-plasma treated gelatin sponges; and

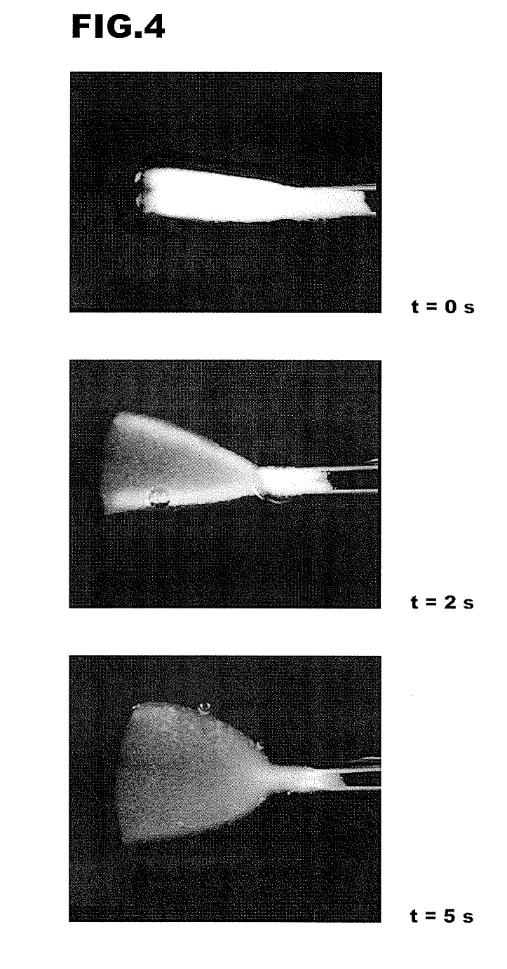

FIG. 4 is a photographic representation of the swelling of a highly compressed gelatin sponge.

### EXAMPLES

### Example 1

### Production of a Quickly Wettable Gelatin Sponge

This example describes the use of the method according to the invention for the production of a quickly wettable material containing hydrocolloid in the form of a gelatin sponge.

Commercially available gelatin sponges that are used as wound dressing were used as starting material. As a result of cross-linkage of the gelatin these sponges are insoluble in water, but are degradable under physiological conditions.

The individual gelatin sponges, which have the original dimensions of 80×50×10 mm, were rolled to a thickness of 4 mm in a first step, so that dimensions of 80×50×4 mm were obtained. As a result of this compression of the gelatin sponges by 60% in one direction, the cellular structure was partially broken open to allow the highest possible efficiency of the subsequent plasma treatment with respect to the entire inside surface of the sponges.

The sponges were then dried in a vacuum (<1 mbar) for 2 hours at 50° C. to reduce the water content of the gelatin, which generally lies at approximately 8 to approximately 12% by weight, as far as possible, e.g. to 2-7% by weight.

70 rolled and dried gelatin sponges with the dimensions 80×50×4 mm were treated with an O₂ plasma in the plasma chamber (volume 301) of a low-pressure plasma plant with high-frequency power supply at 40 kHz for a period of 30 minutes. The machine parameters were set in accordance with the following Table 1 in this case:

TABLE 1

| Parameter | Desired Value |
|---|---|
| Evacuation pressure | 0.4 mbar |
| Maximum evacuation period | 10 min |
| Gas flow component | 100% O₂ |
| Maximum permissible gas deviation | 10% |
| Stabilisation time | 1 min |
| Process pressure | 0.5 mbar |
| Maximum permissible pressure deviation | +/−0.25 mbar |
| Power | 1440 W |
| Maximum permissible power deviation | 15% |
| Process duration | 30 min |
| Rinsing time | 1 min |
| Ventilation time | 1 min |

The gelatin sponges were positioned during the plasma treatment so that they were evenly exposed to the plasma on all six sides.

Pure white sponges with low wetting times were obtained.

### Example 2

### Determination of the Initial Wettability

For the quantitative determination of the wettability of the plasma-treated gelatin sponges according to the invention, the required time for the absorption of a specific quantity of an aqueous solution by the dry sponge was measured (initial wettability).

The procedure of the method is shown schematically in FIG. 1. 50 μl of a PBS buffer with 0.03% by weight of methylene blue (10) were placed in each case on a point of the surface of the sponge (14) using a pipette (12). The period of time from the placement of the drop (t=0) to the complete penetration thereof into the sponge (t=x) was measured. The end point was determined visually by determination of the sponge structure underneath the penetrating drop (16).

At least three gelatin sponges were respectively examined and the measurement was conducted at four to six points of the surface in each sponge. A mean value was formed from all these measurements.

For the gelatin sponges produced according to Example 1, an average value for the initial wettability of 4.4 seconds with a standard deviation of 1.7 seconds was obtained directly after the plasma treatment.

The measurement was repeated after the plasma-treated sponges were stored in atmospheric conditions for 24 hours. In this case, the sponges dried before the plasma treatment again absorbed a certain quantity of moisture. In this case, an initial wettability of 3.6 seconds on average with a standard deviation of 0.9 seconds was measured.

In contrast hereto, the initial wettability of the untreated gelatin sponges serving as starting material in Example 1 was so low that the drop of PBS buffer placed onto the surface had not even penetrated into the sponge after 20 minutes.

This example impressively shows that the wettability of gelatin sponges is significantly improved as a result of the plasma treatment according to the invention. This is a substantial advantage for the medical application of such sponges, as is shown in the following examples.

### Example 3

### Determination of the Blood Absorption Capacity

In this example, the blood absorption capacity of a gelatin sponge according to the invention (produced in accordance with Example 1) was determined quantitatively compared to the non-plasma-treated starting product.

For this, the dry sponges were each weighed and then laid on the surface of a blood sample (human blood with a haematocrit of approximately 50%) for 10 seconds. The sponges were then laid on a filter paper for 60 seconds to allow excess blood to flow off. The sponges with the absorbed blood were weighed once again and the quantity of absorbed blood in relation to the weight of the sponge material was calculated on the basis of the difference from the original weight. The mean value was respectively formed from three measurements.

A clear difference between the two sponges was also evident in this test: while the plasma-treated gelatin sponge absorbed approximately 58-times its own weight in blood, the corresponding value in the case of the dry untreated gelatin sponge amounted to approximately 1.0-times its own weight, which is nearly negligible for practical application.

The quick wettability of the gelatin sponge according to the invention therefore results in a substantially higher blood absorption capacity when the dry sponge is brought into contact with blood.

The rate at which the blood is sucked in by the gelatin sponge (against gravity) was determined in a further test for blood absorption capacity.

The untreated sponge as well as the plasma-treated sponge according to Example 1 were also used here, i.e. both dry and in a moistened or wetted state. To moisten the sponges, these were immersed completely in water before conducting the test and then squeezed out.

The procedure of the method is shown schematically in FIG. 2.

The sponges (20) under investigation each had a length of 80 mm, a width of 20 mm and a thickness of 10 or 4 mm (see Example 1). The sponges (dry or moistened) were laid on a ruler (22) so that the graduation of the ruler ran along the 80 mm long side of the sponge. The ruler was held with the sponge at an angle of 25° to the horizontal and the lower end of the sponge (i.e. the 20 mm wide side) was immersed in a blood concentrate (haematocrit of approximately 50%) diluted with 0.9% by weight of sodium chloride solution (24). Starting with the time of immersion the rise of the blood in the gelatin sponge was measured as a function of time, i.e. the distance that the blood had covered as a result of absorption in the sponge was read on the basis of the ruler scale.

The result of this test is shown in FIG. 3. This shows a graph, in which the rise height of the blood is shown in cm as a function of time in seconds for the four gelatin sponges investigated.

In the case of the dry, non-plasma-treated sponge (curve 1) no detectable absorption of the blood occurred in the sponge over the entire observation period of 5 minutes. If this sponge was moistened beforehand (curve 2), a blood absorption to a rise height of barely 1 cm occurred during the first three minutes and then remained constant. This shows that because of its poor wettability, the non-plasma-treated gelatin sponge must be moistened beforehand to show any absorption action at all.

In contrast, the gelatin sponge plasma-treated using the method according to the invention shows a significantly quicker absorption of the blood, i.e. a higher absorbency, both in the moist state (curve 3) and in the dry state (curve 4). In both cases, during the observation period the blood rose continuously to a height of approximately 3 cm, i.e. to a height of more than 3-times that of the moist, non-plasma-treated sponge.

While the gelatin sponge according to the prior art must be moistened beforehand to be able to be used at all for absorbing blood (i.e. as wound dressing, for example), this is not necessary with the gelatin sponge according to the invention, since this already exhibits a significantly higher absorption effect or absorbency in the dry state.

### Example 4

### Examination of the Haemostatic Effect

The haemostatic (haemorrhage-stopping) effect of the plasma-treated gelatin sponge according to Example 1 and also of the non-plasma-treated comparison material (GELITA-SPON) was examined using a validated ex vivo vein model under standardised conditions.

In this case, a closed off section of the great saphenous vein obtained by varectomy was filled with blood and a diffuse haemorrhage was caused by defined puncture. The blood pressure in the vein was kept constant at 30 mm Hg during the entire test. After a haemorrhaging time of 5 seconds haemostasis was initiated by applying the respective gelatin sponge to the puncture.

Both the time to complete stoppage of the haemorrhaging and the blood loss were detected.

As a result of several of tests, an average time up to complete haemostasis of approximately 5.5 minutes with an average blood loss of approximately 8 ml resulted for the non-plasma-treated gelatin sponge. In contrast, the plasma treatment of the gelatin sponge according to the invention led to a substantial reduction of the average haemostasis time to approximately 3 minutes and to a halving of the average blood loss to approximately 4 ml.

The test shows that the haemostatic effect of gelatin sponges as a result of the plasma treatment according to the invention can surprisingly be significantly improved.

### Example 5

### Production of Quickly Wettable, Highly Compressed Gelatin Sponges

The gelatin sponges investigated in this example were produced in the following manner:

A 15% by weight solution of pigskin gelatin (300 g of bloom) was produced by firstly swelling the gelatin in water and then dissolving it at 60° C. The solution was degassed by means of ultrasound and set to a pH value of 7.2 with 1 mole of sodium hydroxide solution. A corresponding quantity of an aqueous 1% by weight formaldehyde solution was added as cross-linking agent, so that 2000 ppm of formaldehyde (in relation to the gelatin) were provided.

The solution was temperature-controlled to approximately 45° C. after a reaction time of 5 minutes and foamed with air by machine. The foamed gelatin solution, which had a wet density of 120 g/l, was poured into a form with a dimension of 40×20×6 cm and at a relative air humidity of 10% was firstly dried for 2 hours at 20° C. and then for 5 to 7 days at 26° C.

Three different samples were cut out of the dried gelatin sponge with the following dimensions (dimensions axbxc):

Sample 1: 40×150×35 mm

Sample 2: 50×150×35 mm

Sample 3: 80×150×35 mm

All three samples were then subjected to an intense compression in one spatial direction, wherein the dimension c (previously 35 mm) was reduced by rolling to 10 mm in the case of samples 1 and 3 and to 15 mm in the case of sample 2. The sponges were then cut once again to dimension b (previously 150 mm) so that samples with the following dimensions (desired values) were obtained:

Sample 1: 40×7×10 mm

Sample 2: 50×5×15 mm

Sample 3: 80×10×10 mm

The treatment of the three different sponge samples with an O₂ low-pressure plasma was conducted as described in Example 1, but in contrast to the machine parameters specified there, with an evacuation pressure of 0.15 mbar and a process pressure of 0.2 mbar.

### Example 6

### Wetting and Expansion Behaviour of the Highly Compressed Gelatine Sponges

The compressed and plasma-treated gelatin sponges processed in accordance with Example 5 were immersed in water, which led to a rapid wetting and swelling of the sponges. In this case, the swelling time, the dimensions of the sponges before and after swelling and the increase in volume were measured. Two examples of each sample were examined. The results are shown in the following Table 2:

TABLE 2

| | | | Dimensions | | Increase |
|---|---|---|---|---|---|
| | | Swelling | before | Dimensions after | in |
| | | Time | Swelling (mm) | Swelling (mm) | Volume |
| Sample | 1-1 | 5 seconds | 40 × 7 × 11 | 42 × 6 × 40 | 227% |
| | 1-2 | 4 seconds | 40 × 7 × 11 | 42 × 7 × 40 | 282% |
| Sample | 2-1 | 6 seconds | 50 × 5 × 16 | 53 × 5 × 39 | 158% |
| | 2-2 | 6 seconds | 50 × 5 × 16 | 53 × 4 × 40 | 112% |
| Sample | 3-1 | 4 seconds | 79 × 9 × 11 | 82 × 9 × 39 | 268% |
| | 3-2 | 5 seconds | 79 × 9 × 12 | 83 × 10 × 42 | 309% |

These results show that the plasma-treated gelatin sponges are not only wetted very quickly upon contact with liquid, but also swell and expand their volume to a considerable degree. Increases in volume of significantly above 200%, even above 300% in some instances, can be achieved with corresponding compression of the gelatin sponges.

The swelling of a highly compressed gelatin sponge during immersion in water is shown photographically in FIG. 4. The individual images show the sponge directly before immersion (t=0 s), as well as 2 seconds or 5 seconds after immersion. In this case, the sponge is held on the right side by tweezers, which prevents this area from swelling. In contrast, the significant increase in volume in the left area of the sponge is clearly evident within a short time.

Such highly expanded gelatin sponges are particularly suitable for use as nasal plugs, for example.

### Example 7

### Production of Quickly Wettable Gelatin Nonwovens

Matted fibre scrims composed of gelatin fibres (gelatin nonwovens) were produced using the rotary spin process described in the German patent application No. 10 2007 011 606.

The starting point for the production was a 20% by weight aqueous solution of pigskin gelatin (300 g of bloom), which was spun to form a matted scrim composed of gelatin fibres by means of a rotary spinning device, as is also described, for example, in DE 10, 2005 048, 939 A1. Multiple layers of the matted scrim were laid flat one on top of the other to form a gelatin nonwoven with a thickness of approximately 1.7 mm.

The gelatin in the nonwoven was then cross-linked by subjecting this to the equilibrium vapour pressure of a 10% by weight formaldehyde solution for 17 hours. The nonwoven was then kept at approximately 50° C. and approximately 70% relative air humidity for 48 hours to complete the cross-linkage reaction and remove the excess proportion of unconsumed formaldehyde.

Pieces with a dimension of 20×20 mm were cut out of the gelatin nonwoven and subjected to a plasma treatment according to the invention. This was conducted as described in Example 1, but in contrast to the machine parameters specified there, at a process pressure of 0.2 mbar and with a process time of 15 minutes.

When the dry, plasma-treated gelatin nonwoven was brought into contact with human blood, an abrupt wetting and absorption of the blood by the nonwoven occurred. In this case, the plasma-treated nonwoven was completely saturated within less than 1 second, whereas an untreated nonwoven with the same dimensions was only saturated after 45 seconds. Gelatin nonwovens can be used in the medical field for the promotion of haemostasis and/or for wound care in a similar way to the above-described gelatin sponges.

1. A method for the production of a quickly wettable material containing natural hydrocolloid in the form of a shaped body, comprising exposing a material containing natural hydrocolloid in the form of a shaped body to a plasma.

2. The method according to claim 1, wherein the hydrocolloid is selected from the group comprising collagen, gelatin, casein, whey proteins, hyaluronic acid, starch, cellulose, pectins, carrageens, chitosan, agar, alginates, hydrolysates thereof, derivatives thereof as well as mixtures with one another.

3. The method according to claim 1, wherein the material comprises more than approximately 50% by weight of hydrocolloid.

4. The method according to claim 1, wherein the material is composed substantially completely of hydrocolloid.

5. The method according to claim 1, comprising drying the material containing hydrocolloid before exposing it to the plasma.

6. The method according to claim 1, wherein the plasma is a low-pressure plasma.

7. The method according to claim 6, wherein the low-pressure plasma has a pressure of approximately 1 mbar or less.

8. The method according to claim 1, wherein the plasma is an O₂ plasma and/or H₂O plasma.

9. The method according to claim 1, wherein the material is exposed to the plasma for longer than approximately 1 minute.

10. The method according to claim 1, comprising exposing the entire surface of the material containing hydrocolloid to the plasma.

11. The method according to claim 1, wherein the hydrocolloid comprises gelatin.

12. The method according to claim 11, wherein the gelatin is chemically modified.

13. The method according to claim 11, wherein the gelatin is at least partially cross-linked.

14. The method according to claim 11, wherein the material containing gelatin has a cellular structure.

15. The method according to claim 14, wherein the cellular structure has an average pore diameter of less than approximately 300 μm.

16. The method according to claim 14, comprising mechanically compressing the material.

17. The method according to claim 16, comprising compressing the material before exposing the material to the plasma.

18. The method according to claim 16, comprising compressing the material in at least one direction by approximately 40% or more.

19. The method according to claim 18, comprising compressing the material in at least one direction by approximately 65% or more.

20. The method according to claim 11, wherein the material containing gelatin is provided in the form of a film.

21. The method according to claim 20, wherein the film has a thickness of approximately 20 to approximately 500 μm.

22. The method according to claim 11, wherein the material containing gelatin is present in the form of a fibre matting.

23. The method according to claim 22, wherein the fibres have an average thickness in the region of approximately 1 to approximately 500 μm.

24. The method according to claim 11, wherein the material comprises one or more pharmaceutical active substances.

25. The method according to claim 11, wherein the material is coloured.

26. A quickly wettable material containing natural hydrocolloid in the form of a shaped body obtained by the method according to claim 1.

27. A quickly wettable material containing hydrocolloid with a cellular structure, wherein the material has such an initial wettability that is absorbs 50 μl of an aqueous medium within 10 seconds at maximum.

28. The material according to claim 27, wherein the material has a swelling capacity such that, when immersed in an aqueous medium it increases its volume by at least 200% within 6 seconds at maximum.

29-35. (canceled)
